(19) United States
(12) Patent Application Publication
(10) Pub. No.: US 2017/0354588 A1
(43) Pub. Date: Dec. 14, 2017

(54) LONGWEARING COMPOSITION FOR KERATINOUS FIBER

(71) Applicants: Julianne Sortino, Clark, NJ (US); Dhaval Patel, Baskin Ridge, NJ (US); Rossana Lanza, Glen Rock, NJ (US)

(72) Inventors: Julianne Sortino, Clark, NJ (US); Dhaval Patel, Baskin Ridge, NJ (US); Rossana Lanza, Glen Rock, NJ (US)

(21) Appl. No.: 15/180,493

(22) Filed: Jun. 13, 2016

(51) Int. Cl. A61K 8/92; A61K 8/23; A61K 8/49; A61K 8/26; A61K 8/46; A61K 8/81; A61K 8/06; A61K 8/44; A61K 8/97; A61K 8/60; A61K 8/36; A61K 8/368; A61K 8/19; A61K 8/42; A61K 8/65; A61K 8/41; A61Q 1/10; A61K 8/55; A61K 8/73; A61K 8/25; A61K 8/34

(52) U.S. Cl. CPC A61K 8/922; A61K 8/345; A61K 8/342; A61K 8/23; A61K 8/4973; A61K 8/34; A61K 8/26; A61K 8/463; A61K 8/8147; A61K 8/062; A61K 8/44; A61K 8/97; A61K 8/60; A61K 8/361; A61K 8/368; A61K 8/19; A61K 8/42; A61K 8/65; A61K 8/41; A61Q 1/10; A61K 8/55; A61K 8/498; A61K 8/731; A61K 8/25; A61K 8/925; A61K 2800/20

(57) ABSTRACT

Inventive embodiments disclosed herein include an oil-in-water mascara formulation that includes non-emulsifying waxes in a concentration of 1 to 12 percent by weight; at least one viscosity modifier; and at least one non-wax grafted polymer and at least one wax grafted polymer with a combined concentration of 0.10 percent to 50 percent.

### FIELD

Inventive subject matter disclosed herein relates to mascara formulation embodiments.

### BACKGROUND

Conventional oil-in-water mascara formulations typically include high concentrations of non-emulsifying waxes and low concentrations of adhesive, long wearing polymers to create a lash thickening, volumizing mascara. However, these mascara formulations typically do not wear well. They tend to flake off shortly after application.

Also, oil-in-water mascaras with a high load of polymers added for long wear “dilute” the bulk sufficiently to negatively influence and inhibit the volumizing effect of the mascara on the lashes. Usually, with high levels of film forming agent, the mascara bulk has a less creamy texture, thus reducing the building effect on the lashes, imparting a low volumizing effect. Additionally, conventional mascara formulations feel dry, hard and brittle and tend to look dull. To compensate for the low volumizing effect, a high load of waxes is typically employed to add structure and body to the mascara formulation and to provide a desired volume. With increased concentrations of waxes, however, higher concentrations of emulsifier are needed, all of which negatively impact long-lastingness of the mascara film.

### SUMMARY

Inventive embodiments disclosed herein include an oil-in-water mascara formulation that includes non-emulsifying wax(es) in a concentration of 0.10 to 12.00 percent by weight; at least one viscosity modifier; and at least one non-wax grafted polymer and at least one wax grafted polymer with a combined concentration of 0.10 percent to 50.00 percent.

Inventive embodiments disclosed herein include a particular mascara formulation embodiment.

The formulation embodiment includes the following ingredients:

STAGE 1

| | |
|---|---|
| CERA CARNAUBA/COPERNICIA CERIFERA (CARNAUBA) | 2.00% |
| WAX/CIRE DE CARNAUBA | |
| BEESWAX | 8.00% |
| STEARIC ACID | 1.00% |
| CETYL ALCOHOL | 2.00% |
| VP/EICOSENE COPOLYMER | 1.00% |

STAGE 2

| | |
|---|---|
| WATER | QS |
| HECTORITE | 0.30% |
| HYDROXYETHYLCELLULOSE | 0.20% |
| PROPYLENE GLYCOL | 3.00% |
| WATER (and) DISODIUM EDTA | 0.10% |

STAGE 3

| | |
|---|---|
| WATER (and) TETRASODIUM EDTA | 0.12% |

STAGE 4

| | |
|---|---|
| IRON OXIDES | 2.00% |
| IRON OXIDES | 5.00% |
| WATER (and) POLYSORBATE 20 | 1.60% |

STAGE 5

| | |
|---|---|
| AMINOMETHYL PROPANEDIOL | 0.30% |

STAGE 6

| | |
|---|---|
| HYDROGENATED PALM GLYCERIDES (and) POTASSIUM | 2.60% |
| CETYL PHOSPHATE | |

STAGE 7

| | |
|---|---|
| PHENOXYETHANOL | 0.80% |
| WATER (and) PANTHENOL (and) PANTOLACTONE | 0.80% |
| TOCOPHERYL ACETATE | 0.20% |

STAGE 8

| | |
|---|---|
| AMMONIUM ACRYLATES COPOLYMER (AND) WATER | 30.00% |
| (AND) ALCOHOL DENAT. (AND) DISODIUM DECETH-6 | |
| SULFOSUCCINATE (AND) LAURETH-30 (AND) SODIUM | |
| DEHYDROACETATE | |
| ETHYLENE/ACRYLIC ACID COPOLYMER and | 20.00% |
| STYRENE/ACRYLATES COPOLYMER and WATER and | |
| PHENOXYETHANOL and CAPRYLYL GLYCOL and SODIUM | |
| LAURETH-12 SULFATE and C11-15 PARETH-7 and C11-5 | |
| PARETH-40 | |

One other mascara formulation embodiment is as follows:

| | |
|---|---|
| | Concentration |
| INCI Nam | Range (wt. %) |

Phase A

| | |
|---|---|
| Copernicia Cerifera (Carnauba) Wax/CIRE DE | 0.1-12 |
| Carnauba | |
| Beeswax | 6.4-9.6 |
| Stearic Acid | 1-20 |
| Cetyl Alcohol | 0.1-20 |
| VP/Eicosene Copolymer | 2.4-3.6 |

Phase B

| | |
|---|---|
| Water | QS |
| Hectorite | 0.1-5 |
| Hydroxyethylcellulose | 0.1-5 |
| Propylene Glycol | 2.4-3.6 |
| Water and Disodium EDTA | 0.08-0.12 |

Phase C

| | |
|---|---|
| Water and Tetrasodium EDTA | 0.096-0.144 |

Phase D

| | |
|---|---|
| Iron Oxides | 0.1-15 |
| Water and Polysorbate 20 | 1.28-1.92 |

Phase E

| | |
|---|---|
| Aminomethyl Propanediol | 0.24-0.36 |

Phase F

| | |
|---|---|
| Hydrogenated Palm Glycerides (and) Potassium Cetyl | 0.1-20 |
| Phosphate | |

Phase G

| | |
|---|---|
| Phenoxyethanol | 0.64-0.96 |
| Water (and) Panthenol (and) Pantolactone | 0.64-0.96 |

Phase H

| | |
|---|---|
| Ammonium Acrylates Copolymer (and) Water and | 15-30 |
| Alcohol Denat. (and) Disodium Deceth-6 | |
| Sulfosuccinate (and) Laureth-30 (and) Sodium | |
| Dehydroacetate | |
| Ethlene/Acrylate Acid Copolymer and | 5-20 |
| Styrene/Acrylates Copolymer and Water and | |
| Phenoxyethanol And Caprylyl Glycol and Sodium | |
| Laureth-12 Sulfate and C(11-15) Pareth-7 and C(11-5) | |
| Pareth040 | |

### DETAILED DESCRIPTION

The following detailed description includes embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense, and the scope of the present invention is defined by the appended claims and their equivalents.

In this document, the terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Inventive subject matter disclosed herein includes a volumizing mascara formulation that provides pristine wear and easy removal with soap and water as well as with warm water alone. Mascara formulation embodiments disclosed herein include an optimized combination of film formers and waxes that form a flexible film that instantly builds volume and length when applied to a user's lashes and stays put, on a user's lashes, even under humid conditions, while being easy to remove with soap and water as well as with warm water alone. By “stays put” is meant that the mascara does not flake or transfer from a user's lash to another surface.

One particular mascara formulation embodiments as follows:

STAGE 1

| | |
|---|---|
| CERA CARNAUBA/COPERNICIA CERIFERA | 2.00% |
| (CARNAUBA) WAX/CIRE DE CARNAUBA | |
| BEESWAX | 8.00% |
| STEARIC ACID | 1.00% |
| CETYL ALCOHOL | 2.00% |
| VP/EICOSENE COPOLYMER | 3.00% |

STAGE 2

| | |
|---|---|
| WATER | QS |
| HECTORITE | 0.30% |
| HYDROXYETHYLCELLULOSE | 0.20% |
| PROPYLENE GLYCOL | 3.00% |
| WATER (and) DISODIUM EDTA | 0.10% |

STAGE 3

| | |
|---|---|
| WATER (and) TETRASODIUM EDTA | 0.12% |

STAGE 4

| | |
|---|---|
| IRON OXIDES | 2.00% |
| IRON OXIDES | 5.00% |
| WATER (and) POLYSORBATE 20 | 1.60% |

STAGE 5

| | |
|---|---|
| AMINOMETHYL PROPANEDIOL | 0.30% |

STAGE 6

| | |
|---|---|
| HYDROGENATED PALM GLYCERIDES (and) | 2.60% |
| POTASSIUM CETYL PHOSPHATE | |

STAGE 7

| | |
|---|---|
| PHENOXYETHANOL | 0.80% |
| WATER (and) PANTHENOL (and) PANTOLACTONE | 0.80% |
| TOCOPHERYL ACETATE | |

STAGE 8

| | |
|---|---|
| AMMONIUM ACRYLATES COPOLYMER (AND) | 30.00% |
| WATER (AND) ALCOHOL DENAT. (AND) DISODIUM | |
| DECETH-6 SULFOSUCCINATE (AND) LAURETH-30 | |
| AND) SODIUM DEHYDROACETATE | |
| ETHYLENE/ACRYLIC ACID COPOLYMER and | 20.00% |
| STYRENE/ACRYLATES COPOLYMER and WATER and | |
| PHENOXYETHANOL and CAPRYLYL GLYCOL and | |
| SODIUM LAURETH-12 SULFATE and C11-15 PARETH- | |
| 7 and C11-5 PARETH-40 | |

The particular mascara formulation and other inventive embodiments disclosed herein include an oil-in-water mascara formulation that includes a non-emulsifying wax in a concentration of 0.10 to 12.00 percent by weight; at least one viscosity modifier; and at least one non-wax grafted polymer and at least one wax grafted polymer with a combined concentration of 0,1 percent to 50 percent. While the ammonium acrylates copolymer formulation concentration is listed as 30.00% by weight and the styrene/acrylates copolymer formulation is listed as 20.00% by weight above, it is understood that other concentration ranges are suitable so long as the total concentration of these two formulations is 50% of the mascara formulation.

These ingredients form a strong, yet volumizing film, which eliminates the need for very high concentrations of waxes in the formula. Mascara formulation embodiments also include 12% or less of non-emulsifying wax in an oil phase, about 50% less than what is used conventionally in high volume washable mascara formulations. This combination of ingredients work in synergy, to form a durable and long lasting, yet high volumizing film, which is easily removed with soap and water or warm water alone. Some embodiments are easily removed with soap and water. Other embodiments are easily removed with warm water alone.

Inventive mascara embodiments directly solve a problem of making a long lasting mascara that is also easily removable with soap and water or with warm water alone, without soap.

Another exemplary embodiment of a long wearing mascara formulation is as follows:

| | |
|---|---|
| | Concentration |
| INCI Nam | Range (wt. %) |

Phase A

| | |
|---|---|
| Copernicia Cerifera (Carnauba) Wax/CIRE DE | 0.1-12 |
| Carnauba | |
| Beeswax | 6.4-9.6 |
| Stearic Acid | 0.1-20 |
| Cetyl Alcohol | 0.1-20 |
| VP/Eicosene Copolymer | 2.4-3.6 |

Phase B

| | |
|---|---|
| Water | QS |
| Hectorite | 0.1-5 |
| Hydroxyethylcellulose | 0.1-5 |
| Propylene Glycol | 2.4-3.6 |
| Water and Disodium EDTA | 0.08-0.12 |

Phase C

| | |
|---|---|
| Water and Tetrasodium EDTA | 0.096-0.144 |

Phase D

| | |
|---|---|
| Iron Oxides | 0.1-15 |
| Water and Polysorbate 20 | 1.28-1.92 |

Phase E

| | |
|---|---|
| Aminomethyl Propanediol | 0.24-0.36 |

Phase F

| | |
|---|---|
| Hydrogenated Palm Glycerides and Potassium Cetyl | 0.1-20 |
| Phosphate | |

Phase G

| | |
|---|---|
| Phenoxyethanol | 0.64-0.96 |
| Water (and) Panthenol (and) Pantolactone | 0.64-0.96 |

Phase H

| | |
|---|---|
| Ammonium Acrylates Copolymer (and) Water (and) | 15-30 |
| Alcohol Denat. (and) Disodium Deceth-6 | |
| Sulfosuccinate (and) Laureth-30 (and) Sodium | |
| Dehydroacetate | |
| Ethylene/Acrylate Acid Copolymer and | 5-20 |
| Styrene/Acrylates Copolymer and Water and | |
| Phenoxyethanol And Caprylyl Glycol and Sodium | |
| Laureth-12 Sulfate and C(11-15) Pareth-7 and C(11-5) | |
| Pareth040 | |

While hectorite and hydroxymethylcellulose are disclosed as aqueous viscosity modifiers, it is believed that other aqueous viscosity modifiers may be usable in formulations disclosed herein, either singly or in combination.

While stearic acid and cetyl alcohol are disclosed herein as emulsifiers, it is believed that other emulsifiers including hydrogenated palm glycerides and potassium cetyl phosphate, and polysorbate 20, and other conventional emulsifiers are also suitable emulsifiers used singly or in combination.

While black iron oxide is disclosed herein, it is believed that other pigments are usable in formulation embodiments disclosed herein. Specific black iron oxide formulations employed include Black Iron Oxide C33-5000 and black iron oxide C33-6000, both manufactured by Sun Chemical.

While aminomethyl propandiol is disclosed herein, it is believed that other conventional pH adjusters and buffers are usable for use herein.

For some embodiments, the concentration of ingredients in Phase exceeds 50% by weight.

The term, “QS” used to describe the concentration of water refers to the amount of water added to take the formulation to 100% by weight.

Inventive embodiments disclosed herein additionally include film formers. In the formulation embodiment disclosed herein, the polymer combination includes at least one polyolefin grafted film former and one or more acrylates type film former, in a combined concentration of 0.1-50%.

In the exemplary formulation embodiment disclosed herein, a non-emulsifying wax is Carnauba wax, listed in Stage 1. Carnauba wax is also known as Carnauba Wax Prime, Yellow 2442, manufactured by Kahl GmbH &Co. Another wax is the 8104 Kahl White Beeswax, manufactured by Kahl GmbH & Co. The stearic acid and cetyl alcohol (Lanette 16) are emulsifiers that enable stage 1 ingredients to blend with aqueous based ingredients in Stage 2. VP/Eicosene copolymer, sold as Unimer U-15, manufactured by Induchem USA, is alkylated vinyl pyrolidone copolymer. In the formulation embodiments disclosed herein, the alkylated vinyl pyrolidone acts as a film former and pigment dispersing agent.

Stage 2 is an aqueous stage with water, a powdered smectite clay, hectorite, sold as Bentone EW, manufactured by Elementis Specialties; hydroxyethyl cellulose, sold as NATROSOL 250 HR PC, manufactured by Ashland Inc. Stage 2 also includes propylene glycol and water.

The mascara pigments, black iron oxide, are ingredients in Stage 4 of the formulation.

Stage 5 includes the aminomethyl propanediol, sold as MPD Ultra-PC, that acts to neutralize acids in the formulation to form salts and water.

The phenoxyethanol of Stage 7 is a germicide. D-Panthenol 75L, manufactured by Coptis, is a moisturizer. Vitamin E is an anti-oxidant.

For some embodiments, other waxes are used in conjunction with non-emulsifying waxes. The low concentrations of non-emulsifying waxes provide a flexible formulation, while also adding to the building characteristics of the formula by providing structure and slip necessary to aid in volumizing benefit of mascara.

Volumizing mascara embodiments disclosed herein provide pristine wear and easy removal from eyelashes with soap and water for some embodiments or warm water alone as described herein. Formulation embodiments disclosed herein provide an optimized combination of film formers and waxes producing a flexible film that instantly build volume and length and stays put through humid conditions while being easy to remove.

The combination of hectorite, the Bentone smectite clay, and hydroxyethylcellulose act as an aqueous viscosity modifying agent, which in turn, facilitates deposition of the formulation embodiments onto lashes.

Emulsifiers are used in both the oil and water phases to keep the two phases together and to prevent phase separation by forming a tight, stable oil-in-water emulsion.

The combination of two non-wax grafted and wax grafted polymers at a combined concentration of 0.1%-50% aid to form a durable, flexible, and volumizing film that provides additional structure to the formulation as well. Examples of such polymers are formulations that include ethylene/acrylate acid copolymer and styrene/acrylates copolymer and water and phenoxyethanol and caprylyl glycol and sodium laureth-12 sulfate and C(11-15) pareth-7 and C(11-5) pareth-40, with a formulation of ammonium acrylates copolymer, water, alcohol, denatured, disodium deceth-6 sulfosuccinate, laureth-30 and sodium.

By using varying concentration ratios of both a formulation that includes ethylene/acrylate acid copolymer and styrene/acrylates copolymer and water and phenoxyethanol and caprylyl glycol and sodium laureth-12 sulfate and C(11-15) pareth-7 and C(11-5) pareth-40, and a formulation of ammonium acrylates copolymer, water, alcohol, denatured, disodium deceth-6 sulfosuccinate, laureth-30 and sodium of Stage 8, in combination with 0.10-12.00% wax(es), and aqueous viscosity modifying agents, the mascara features of longevity on a lash and volumizing on the lash are achieved. The volumizing mascara formula employs a high concentration of polymer and low concentration of waxes, contrary to known oil in water mascara formulations.

In the embodiments presented herein, the formulation of ammonium acrylates copolymer, water, alcohol, denatured, disodium deceth-6 sulfosuccinate, laureth-30 and sodium has a concentration of 25.00% of the mascara formulation. The formulation that includes ethylene/acrylate acid copolymer and styrene/acrylates copolymer and water and phenoxyethanol and caprylyl glycol and sodium laureth-12 sulfate and C(11-15) pareth-7 and C(11-5) pareth-40, has a concentration of 15.00% of the mascara formulation. The formulation of ammonium acrylates copolymer, water, alcohol, denatured, disodium deceth-6 sulfosuccinate, laureth-30 and sodium is an ammonium acrylates copolymer manufactured by Worlee of Hamburg Germany under the name, Micromer C60/42. The formulation that includes ethylene/acrylate acid copolymer and styrene/acrylates copolymer and water and phenoxyethanol and caprylyl glycol and sodium laureth-12 sulfate and C(11-15) pareth-7 and C(11-5) pareth-40, is manufactured by Interpolymer under the name, Syntran PC 5288, and is formed by grafting an amorphous functional acrylic polymer section onto a crystalline polyethylene backbone.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

What is claimed is:

1. An oil-in-water mascara formulation comprising:
Non-emulsifying waxes in a concentration of 1 to 12 percent by weight;
at least one viscosity modifier in the aqueous phase; and
at least one non-wax grafted polymer and at least one wax grafted polymer with a combined concentration of 0.10 percent to 50 percent.

2. The mascara formulation of claim 1, wherein the wax is a Carnauba wax

3. The mascara formulation of claim 1, wherein the viscosity modifier is smectite clay

4. The mascara formulation of claim 1, wherein the viscosity modifier is hydroxyethycellulose.

5. The mascara formulation of claim 1, wherein the wax-grafted polymer is an ethylene acrylic acid copolymer.

6. The mascara formulation of claim 1, wherein the non-wax grafted polymer is an ammonium acrylates copolymer.

7. The mascara formulation of claim 1 further comprising one or more emulsifiers.

8. The mascara formulation of claim 1, further comprising one or more moisturizers.

9. An oil-in-water mascara formulation, comprising:
STAGE 1

| | |
|---|---|
| CERA CARNAUBA/COPERNICIA CERIFERA (CARNAUBA) | 2.00% |
| WAX/CIRE DE CARNAUBA | |
| BEESWAX | 8.00% |
| STEARIC ACID | 1.00% |
| CETYL ALCOHOL | 2.00% |
| VP/EICOSENE COPOLYMER | 3.00% |

STAGE 2

| | |
|---|---|
| WATER | QS |
| HECTORITE | 0.30% |
| HYDROXYETHYLCELLULOSE | 0.20% |
| PROPYLENE GLYCOL | 3.00% |
| WATER (and) DISODIUM EDTA | 0.10% |

STAGE 3

| | |
|---|---|
| WATER (and) TETRASODIUM EDTA | 0.12% |

STAGE 4

| | |
|---|---|
| IRON OXIDES | 2.00% |
| IRON OXIDES | 5.00% |
| WATER (and) POLYSORBATE 20 | 1.60% |

STAGE 5

| | |
|---|---|
| AMINOMETHYL PROPANEDIOL | 0.30% |

STAGE 6

| | |
|---|---|
| HYDROGENATED PALM GLYCERIDES (and) POTASSIUM | |
| CETYL PHOSPHATE | 2.60% |

STAGE 7

| | |
|---|---|
| PHENOXYETHANOL | 0.80% |
| WATER (and) PANTHENOL (and) PANTOLACTONE | 0.80% |
| TOCOPHERYL ACETATE | 0.20% |
| ARGANIA SPINOSA KERNEL OIL | 0.10% |
| HYDROLYZED KERATIN (and) WATER (and) POTASSIUM | 0.03% |
| SORBATE (and) SODIUM BENZOATE (and) | |
| PHENOXYETHANOL (and) SODIUM SULFATE | |
| COLLAGEN (and) ACRYLATES/CARBAMATE COPOLYMER | 0.03% |

STAGE 8

| | |
|---|---|
| AMMONIUM ACRYLATES COPOLYMER (AND) WATER | 30.00% |
| (AND) ALCOHOL DENAT. (AND) DISODIUM DECETH-6 | |
| SULFOSUCCINATE (AND) LAURETH-30 (AND) SODIUM | |
| DEHYDROACETATE | |
| ETHYLENE/ACRYLIC ACID COPOLYMER and | 20.00% |
| STYRENE/ACRYLATES COPOLYMER and WATER and | |
| PHENOXYETHANOL and CAPRYLYL GLYCOL and SODIUM | |
| LAURETH-12 SULFATE and C11-15 PARETH-7 and C11-5 | |
| PARETH-40 | |

10. An oil-in-water mascara, comprising:

| | |
|---|---|
| | Concentration |
| INCI Nam | Range (wt. %) |

Phase A

| | |
|---|---|
| Copernicia Cerifera (Carnauba) Wax/CIRE DE | 0.1-12 |
| Carnauba | |
| Beeswax | 6.4-9.6 |
| Stearic Acid | 0.1-20 |
| Cetyl Alcohol | 0.1-20 |
| VP/Eicosene Copolymer | 2.4-3.6 |

Phase B

| | |
|---|---|
| Water | QS |
| Hectorite | 0.1-5 |
| Hydroxyethylcellulose | 0.1-5 |
| Propylene Glycol | 2.4-3.6 |
| Water and Disodium EDTA | 0.08-0.12 |

Phase C

| | |
|---|---|
| Water and Tetrasodium EDTA | 0.096-0.144 |

Phase D

| | |
|---|---|
| Iron Oxides | 0.1-15 |
| Water and Polysorbate 20 | 1.28-1.92 |

Phase E

| | |
|---|---|
| Aminomethyl Propanediol | 0.24-0.36 |

Phase F

| | |
|---|---|
| Hydrogenated Palm Glycerides (and) Potassium Cetyl | 0-20 |
| Phosphate | |

Phase G

| | |
|---|---|
| Phenoxy ethanol | 0.64-0.96 |
| Water (and) Panthenol (and) Pantolactone | 0.64-0.96 |

Phase H

| | |
|---|---|
| Ammonium Acrylates Copolymer (and) Water (and) | 15-30 |
| Alcohol Denat. (and) Disodium Deceth-6 | |
| Sulfosuccinate (and) Laureth-30 (and) Sodium | |
| Dehydroacetate | |
| Ethlene/Acrylate Acid Copolymer and | 5-20 |
| Styrene/Acrylates Copolymer and Water and | |
| Phenoxyethanol And Caprylyl Glycol and Sodium | |
| Laureth-12 Sulfate and C(11-15) Pareth-7 and C(11-5) | |
| Pareth040 | |
